(19) United States
(12) Patent Application Publication
(10) Pub. No.: US 2010/0291168 A1
(43) Pub. Date: Nov. 18, 2010

(54) ANTIMICROBIAL FORMULATIONS COMPRISING A COMBINATION OF A PYRIDINE THIOL AND A BIS-QUINOLINIUM SALT

(76) Inventors: Priscila Gottardello, West Yorkshire (GB); Andleeb Qureshi, West Yorkshire (GB)

Correspondence Address: Weaver Austin Villeneuve & Sampson LLP, P.O. BOX 70250, OAKLAND, CA, 94612-0250, US

(21) Appl. No.: 12/663,193

(22) PCT Filed: Jun. 5, 2008
(86) PCT No.: PCT/GB08/01908

§ 371 Date: May 24, 2010

(30) Foreign Application Priority Data
Jun. 7, 2007 (GB) 0710899.6

(51) Int. Cl. A61K 31/4709; A61K 9/12; A61K 8/58; A01N 25/34; A61P 31/02; A61P 17/10; A61Q 15/00

(52) U.S. Cl. 424/404; 514/313; 424/45; 424/65

(57) ABSTRACT

An antimicrobial formulation containing a pyridine thiol and a bis-quinolinium salt which contains a cation of formula (I) below: wherein n is an integer from 3 to 18. The formulation may in particular be used for the treatment of a bacterial condition, more particularly acne or body odour. It is suitably applied topically.

### FIELD OF THE INVENTION

This invention relates to antimicrobial formulations, and to the use of certain combinations of compounds as antimicrobial agents, in particular for the treatment of acne.

### BACKGROUND TO THE INVENTION

Pyrithione, also known as 1-hydroxy-2(1H)-pyridinethione, 2-pyridinethiol-1-oxide or 2-mercaptopyridine N-oxide, is a type of pyridine thiol and is known for use as a bactericide and fungicide. It is typically used in the form of a metal salt (strictly, a chelated complex) such as zinc pyrithione. In particular the zinc salt is known as an antiseborrheic, an antifungal and an antibacterial agent, as well as for the treatment of scalp conditions such as dandruff and as an anti-acne agent. It has also been used as a cosmetic preservative, for inhibiting mould growth on fabrics in commercial laundries and as a preservative (persistence agent) for topical antiseptic formulations. (See Guthery, E. et al, Am. J. Infect. Control 33(1), 2005: 15-22.)

It is also known to use quaternary ammonium compounds, for example benzalkonium chloride, benzethonium chloride, cetalkonium chloride or dequalinium chloride, as antimicrobial agents. For example:
U.S. Pat. No. 3,147,182 describes a synergistic mixture of a dequalinium salt and a cetyl pyridinium salt in a topical antibacterial composition.
U.S. Pat. No. 4,006,218 refers to the use of dequalinium chloride as an antimicrobial agent in a topical antimicrobial composition, in combination with an alcohol, alkanol or phenol “potentiator”.
U.S. Pat. No. 4,176,197 discloses a topical anti-acne composition containing a quaternary ammonium halide together with a thio-amino acid or related compound.
U.S. Pat. No. 4,294,852 describes a topical composition for treating certain skin conditions such as psoriasis and eczema, which can contain a quaternary ammonium active such as dequalinium chloride or benzalkonium chloride.
U.S. Pat. No. 4,474,748 discloses a topical antimicrobial composition containing a synergistic mixture of a quaternary ammonium compound, such as dequalinium chloride or benzalkonium chloride, with a phenyl alkanol potentiator. The composition can be used to treat wounds or as a surgical scrub.
U.S. Pat. No. 6,001,864 discloses a topical antifungal composition containing a quaternary ammonium salt with an imidazole.
U.S. Pat. No. 6,015,816 describes antimicrobial compositions containing quaternary ammonium compounds attached to colloidal particles such as clays.
U.S. Pat. No. 6,423,750 describes the use of quaternary ammonium compounds such as benzalkonium chloride as topical anti-infective agents for the treatment of skin conditions.
US-2006/0019987 describes the use of quaternary ammonium compounds as topical antiviral agents, referring to dequalinium chloride and benzalkonium chloride as candidate actives.
JP-2005-350357 discloses the use of dequalinium chloride as an anti-perspirant and of benzalkonium chloride as a bactericide, in a deodorant formulation.

It has now surprisingly been found that when a pyridine thiol such as zinc pyrithione is combined with certain types of quaternary ammonium compound, a synergistic effect can be observed on their combined level of antimicrobial activity, in particular versus propionibacteria and bacteria of the genus Corynebacterium, and more particularly versus Propionibacterium acnes, the bacterium implicated in inflammatory acne. As a result, novel antimicrobial and/or anti-acne formulations can be prepared, in particular for topical application, either with improved efficacy and/or containing lower levels of at least one of the active ingredients than would previously have been thought necessary.

### STATEMENTS OF THE INVENTION

According to a first aspect of the present invention there is provided an antimicrobial formulation containing (a) a pyridine thiol (in particular a pyrithione or pyrithione derivative) and (b) a bis-quinolinium salt which contains a cation of formula (I) below:

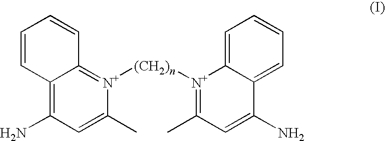

wherein n is an integer from 3 to 18.

This formulation is preferably suitable for topical application to, and/or contact with, the skin, in particular human skin. The pyridine thiol and the bis-quinolinium salt are therefore preferably contained in a pharmaceutically acceptable vehicle which can safely be applied to, and/or contacted with, the skin. A formulation which is “suitable for” topical application may also be adapted for topical application.

Suitable vehicles will be well known to those skilled in the art of preparing topical skin care or pharmaceutical preparations. The vehicle will typically be a fluid, which term includes a cream, paste, gel, lotion, foam, ointment or other viscous or semi-viscous fluid. The pyridine thiol and the bis-quinolinium salt may each independently be present in the form of a solution or suspension, the term “suspension” including emulsions, micellar systems and other multi-phase dispersions as well as suspensions of particles of the relevant substances.

Either or both of the pyridine thiol and the bis-quinolinium salt may, whether separately or together, be carried in or on a delivery vehicle which is suitable for targeting or controlling its release at the intended site of administration. Such vehicles include liposomes and other encapsulating entities, for example niosomes, aspasomes, ethosomes, microsponges, microemulsions, hydrogels and solid lipid nanoparticles.

In the context of the present invention, the pyridine thiol may for example be a 2-pyridine thiol, 3-pyridine thiol or 4-pyridine thiol, in particular a 2- or 4-pyridine thiol. It may be present in the form of a salt or other derivative, for instance a pyridine thiol oxide or hydroxide. It is preferably a pyrithione (ie, an N-oxide pyridine thiol) or tautomer or derivative thereof.

A pyrithione may be present in the form of a pyrithione derivative, eg, a molecular and/or ionic complex containing the pyrithione group, such as for example a pyrithione salt or a dimer, oligomer or polymer containing a pyrithione or pyrithione salt monomer (for example, dipyrithione, also known as di-2-pyridinedisulphide-1,1′-dioxide).

Suitable salts of pyridine thiols, in particular pyrithiones, include metal salts such as zinc, selenium, silver, copper and sodium salts, preferably zinc or copper, most preferably zinc (eg, zinc-2-pyridinethiol-1-oxide).

Thus most preferably the pyridine thiol is present as a pyrithione salt, in particular a metal salt/complex such as are mentioned above. Most preferably it is zinc pyrithione.

The pyridine thiol is preferably pharmaceutically acceptable, which term includes suitable for veterinary use.

A formulation according to the invention may contain a mixture of two or more different pyridine thiols.

The bis-quinolinium salt contains a cation of formula (I) above, in which n is an integer from 3 to 18, together with one or more appropriate counterions. n is suitably an integer from 4 to 15 or from 8 to 12, for example 10.

In a preferred embodiment of the invention, the bis-quinolinium salt is a dequalinium salt, in which n is 10. It may be selected from dequalinium chloride, dequalinium iodide, dequalinium acetate and mixtures thereof.

In the present context, the bis-quinolinium salt will suitably be a pharmaceutically acceptable salt such as a halide (for example a chloride, bromide or iodide, in particular a chloride or iodide) or a carboxylate. Suitable carboxylates include the acetate, salicylate, lactate, citrate, ascorbate, gluconate, laurate, myristate, palmitate, undecenoate and aspirinate—of these, the acetate or the salicylate may be preferred, in particular the acetate.

In an embodiment of the invention, the bis-quinolinium salt is a halide. It may for example be a chloride. It may be an iodide.

The bis-quinolinium salt is preferably pharmaceutically acceptable, which term includes suitable for veterinary use.

A formulation according to the invention may contain a mixture of two or more different bis-quinolinium salts.

Either or both of the pyridine thiol and the bis-quinolinium salt may be replaced, in a formulation according to the invention, by a suitable derivative, in particular a pharmaceutically acceptable (which term includes acceptable for veterinary use, but suitably implies at least acceptability for human pharmaceutical use) derivative. It may be for example a salt, complex or solvate or a so-called “prodrug” form or protected form which reverts to an active form of the relevant compound at an appropriate time on or after administration. In an embodiment, however, the pyridine thiol is not used in the form of a prodrug or other protected form. In an embodiment, the bis-quinolinium salt is not used in the form of a prodrug or other protected form.

In a formulation according to the invention, both the pyridine thiol and the bis-quinolinium salt are ideally present as active (ie, antimicrobially, preferably antibacterially, active) agents. They may be present as anti-acne agents (ie, agents which are active against a symptom and/or a cause of acne and/or against one or more micro-organisms associated with acne). Surprisingly, such agents have been found to act together synergistically to inhibit, and often to prevent, microbial (in particular bacterial, and more particularly propionibacterial) activity. In other words, they have been found to increase one another's activity in a manner which can be synergistic compared to the sum of the activities of the two agents individually.

It is possible that the potentiation of one another's antimicrobial activity by a pyridine thiol and a bis-quinolinium salt may be at least partly due to the formation of a reaction product having an antimicrobial activity greater than the sum of those of the individual reactants. The invention may thus embrace an antimicrobial formulation containing a reaction product formed between a pyridine thiol and a bis-quinolinium salt (for example a dequalinium salt), in particular between zinc pyrithione and a dequalinium salt such as dequalinium chloride or dequalinium iodide; this reaction product may be formed in situ immediately prior to, or at the point of, use.

In a formulation according to the invention the pyridine thiol and the bis-quinolinium salt, and their relative proportions, are preferably such as to yield at least an additive level of antimicrobial or antibacterial activity compared to the activities of the individual compounds alone (this is sometimes referred to as an “indifferent” interaction between the compounds). More preferably, the compounds and their relative proportions are such as to yield a synergistic effect on antimicrobial or antibacterial activity, by which is meant that the antimicrobial or antibacterial activity of the combination of the two compounds is greater than the sum of the individual activities of the same amounts of the two compounds used individually. An increased level of activity in these contexts may be manifested by a lower concentration of the compound(s) being needed to inhibit and/or to kill the relevant micro-organism, and/or by a larger zone of inhibition in a disc diffusion assay, and/or by a faster rate of microbial inhibition or killing.

Antimicrobial activity may be growth inhibitory activity or more preferably biocidal (ie, lethal to the relevant micro-organism). It preferably includes at least antibacterial activity, which can encompass activity against both Gram-positive and Gram-negative bacteria, in particular Gram-positive bacteria. It may comprise activity against sessile and/or planktonic bacteria.

A formulation according to the present invention preferably has antibacterial activity.

In the context of this invention, activity against a particular species of micro-organism may be taken to mean activity against at least one, preferably two or more, strains of that species.

Antimicrobial activity may be or include the ability to disrupt and/or suppress biofilm formation by the relevant organism.

In the present context, the disruption of biofilm formation embraces any negative effect on the ability of a micro-organism to form, maintain or exist in a biofilm, and/or on a biofilm already formed by the organism. Thus, it may involve reducing the amount of a previously formed biofilm, and/or impairing such a biofilm. It may involve killing or inhibiting sessile bacteria within a biofilm.

Suppression of biofilm formation embraces any degree of impairment (including complete prevention) of the ability of a micro-organism to form, or more typically to co-aggregate with, a biofilm. It thus embraces total or partial impairment, including reducing the amount and/or strength of biofilm which the organism is able to form and/or the speed with which it is able to do so. It may involve preventing or reducing the growth or the rate of growth of an existing biofilm formed by the organism.

An antimicrobial formulation according to the present invention is preferably active at least against Gram-positive bacteria, for example against one or more bacteria selected from Propionibacterium spp, staphylococci and bacteria implicated in body odour. It may in particular be active against one or more bacteria selected from Propionibacterium spp and bacteria implicated in body odour, more particularly against one or more of the bacteria referred to below in connection with the fifth to the eighth aspects of the invention.

A formulation according to the present invention is preferably active against bacteria associated with skin or skin-borne infections, in particular acne; it is thus preferably active against propionibacteria and/or other bacteria associated with acne and most preferably against one or more strains of Propionibacterium acnes. It may be active against staphylococci (and in cases other Gram-positive cocci such as enterococci), for example Staphylococcus aureus. It may be active against one or more bacteria associated with body odour, in particular in the axilla or feet; examples of such bacteria include members of the genus Corynebacterium, as described in more detail below in connection with the fifth aspect of the invention.

In a preferred embodiment of the invention, the formulation is active against one or more strains of P. acnes and/or in some instances against one or more strains of P. granulosum.

The formulation is preferably active against micro-organisms, in particular bacteria and more particularly propionibacteria, which are wholly or partially resistant to one or more antibiotics, for instance those which are in common clinical use. It is ideally active against macrolide-lincosamide-streptogramin (MLS) resistant and/or macrolide-lincosamide-streptogramin-ketolide (MLSK) resistant strains of bacteria. In particular it may be active against erythromycin-resistant, clindamycin-resistant and/or tetracycline-resistant strains of bacteria, for example P. acnes strains, the term tetracycline here referring to the class of antibiotics including for example minocycline and doxycycline as well as the specific antibiotic known as tetracycline.

Antimicrobial activity may be measured in conventional manner, for instance using the tests described in the examples below. Generally tests for activity involve treating a culture of the relevant micro-organism with the candidate antimicrobial compound or combination, incubating the treated culture under conditions which would ordinarily support growth of the orgnaism, and assessing the level of growth, if any, which can occur in the presence of the candidate compound or combination.

Preferably the pyridine thiol used in the present invention has a minimum inhibitory concentration (MIC), at least against propionibacteria, of 50 μg/ml or less, more preferably 10 μg/ml or less, most preferably 5 or even 2 μg/ml or less. Its corresponding minimum biocidal concentration (MBC) is preferably 100 μg/ml or less, more preferably 50 mg/ml or less, yet more preferably 10 μg/ml or less. Suitably the ratio of its MIC to its MBC is from 0.01 to 1 or from 0.125 to 1, ideally from 0.5 to 1. More preferably the pyridine thiol also exhibits such characteristics in the presence of at least one of, preferably both of, lipid (for example either Tween™ 80 or triolein at 1% v/v) and salt (sodium chloride)—these are species which can be present at the surface of the skin and hence performance in this context can be indicative of suitability for use in topical skin treatment formulations, in particular in the context of acne treatment.

Preferably the bis-quinolinium salt used in the present invention has a minimum inhibitory concentration (MIC), at least against propionibacteria, of 125 μg/ml or less, more preferably 62.5 or 31.25 or 15.6 or in cases 10 μg/ml or less, such as from 31.25 to 3.9 μg/ml. Its corresponding minimum biocidal concentration (MBC) is preferably 125 μg/ml or less, more preferably 62.5 or 31.25 or even 20 μg/ml or less. Suitably the ratio of its MIC to its MBC is from 0.125 to 1, ideally from 0.5 to 1. Again, the bis-quinolinium salt preferably also exhibits such characteristics in the presence of at least one of, preferably both of, lipid and salt, as described above in connection with the pyridine thiol activity.

MIC and MBC values may be measured using conventional assay techniques, for instance as described in the examples below.

The concentration of the pyridine thiol in the formulation might suitably be 0.01% w/v or greater, preferably 0.05% w/v or 0.1% w/v or greater. Its concentration might be up to 1% w/v, preferably up to 0.5 or 0.25% w/v.

The concentration of the bis-quinolinium salt in the formulation might suitably be 0.05% w/v or greater, preferably 0.1% w/v or greater, more preferably 0.5% w/v or greater. Its concentration might be up to 10 or 5% w/v, preferably up to 2.5% w/v, more preferably up to 2% w/v.

Due to the presence of the other compound, it may be possible for the concentration of either the pyridine thiol or the bis-quinolinium salt, at the site of action when the formulation is applied in vivo, to be less than the MBC, or even than the MIC, of that compound alone. For instance the concentration of at least one of the compounds at this point may be 0.8 or less times its MBC or MIC, such as 0.5 or less, 0.25 or less or 0.125 or less.

Preferably the weight ratio of the pyridine thiol in the formulation to that of the bis-quinolinium salt is from 1:500 to 500:1, more preferably from 1:50 to 50:1 or from 1:10 to 10:1 or from 1:50 to 1:1, yet more preferably from 1:20 to 1:1, most preferably from 1:10 to 1:1 or from 1:5 to 1:1. In an embodiment of the invention, the weight ratio of the pyridine thiol in the formulation to that of the bis-quinolinium salt is from 1:50 to 1:1, or from 1:20 to 1:1, or from 1:10 to 1:1, or from 1:50 or 1:20 or 1:10 to 1:2 or 1:5.

In an embodiment neither the pyridine thiol nor the bis-quinolinium salt is present, in a formulation according to the invention, either purely or even primarily as an antifungal agent.

The formulation of the invention is preferably suitable for, and more preferably adapted for, topical administration to human skin. It may be suitable for, or adapted for, topical administration to other epithelia such as the nares, scalp, ears, eyes or vagina, in particular the nares and/or ears. It may take the form of a lotion, cream, ointment, varnish, foam, paste, gel, suppository or pessary or any other physical form known for topical administration, in particular a lotion, cream, ointment, foam, paste or gel. It may comprise a formulation which is, or may be, applied to a carrier such as a sponge, swab, brush, tissue, cloth, wipe, skin patch or dressing (which includes a bandage, plaster, skin adhesive or other material designed for application to a tissue surface) to facilitate its topical administration. It may take the form of a nasal spray or of eye or ear drops. It may be intended for pharmaceutical (which includes veterinary but is preferably human) use, for example to treat skin infections, or as a prophylactic against infections such as MRSA, and/or for cosmetic or other non-medical care purposes (for example, for general hygiene or skin cleansing).

The vehicle in which the pyridine thiol and the bis-quinolinium salt are contained may be any vehicle or mixture of vehicles which is suitable for topical application; the type chosen will depend on the intended mode and site of application. Many such vehicles are known to those skilled in the art and are readily available commercially. Examples may for instance be found in Williams' “Transdermal and Topical Drug Delivery”, Pharmaceutical Press, 2003, and other similar reference books. See also Date, A. A. et al, Skin Pharmacol. Physiol., 2006, 19(1): 2-16 for a review of topical drug delivery strategies, and also “Skin Delivery Systems”, 2006, John J. Wille, Ed., Blackwell Publishing; “Textbook of Cosmetic Dermatology”, 2004, 3^{rd} edition, Robert Baran, Howard I Maibach, Taylor & Francis; and “Skin Care Beyond the Basics”, 2001, Mark Lees, Milady. Either or both of the active agents may be present in the form of a suspension or other form of multi-phase dispersion, as described above.

Also as described above, the vehicle may be such as to target a desired site and/or time of delivery of the formulation. It may for instance target the formulation to the skin or hair follicles, most preferably to the hair follicles and/or pilosebaceous follicles. It may delay or otherwise control release of the formulation over a particular time period. Either or both of the pyridine thiol and the bis-quinolinium salt may be microencapsulated, for instance in liposomes—particularly suitable liposomes, for topical use, are those made from stratum corneum lipids, eg, ceramides, fatty acids or cholesterol.

In some cases a polar vehicle may be preferred. In an embodiment, the vehicle may be primarily non-aqueous, and is suitably volatile. In another embodiment, the vehicle may be aqueous. In cases the vehicle may be alcohol-based or silicon-based. The vehicle may include a solubilising agent to assist solubilisation of either or both of the pyridine thiol and the bis-quinolinium salt.

By way of example, a lotion or gel formulation may contain a mixture of water, an alcohol such as ethanol or phenoxyethanol and a glycol such as propylene glycol.

The formulation may contain standard excipients and other additives known for use in pharmaceutical formulations, in particular topical skin care formulations. Examples include emollients, perfumes, antioxidants, preservatives, stabilisers, gelling agents and surfactants; others may be found in Williams' “Transdermal and Topical Drug Delivery”, supra. For the treatment of acne, however, it may be preferred for the formulation not to contain an emollient.

Such a formulation may further contain additional active agents. For example, it may contain one or more additional agents selected from anti-acne agents, keratolytics, comedolytics, agents capable of normalising keratinocyte and/or sebocyte function, anti-inflammatories, anti-proliferatives, antibiotics, anti-androgens, sebostatic/sebosuppressive agents, anti-pruritics, immunomodulators, agents which promote wound healing, additional antimicrobial (in particular antibacterial) agents and mixtures thereof. It may in particular contain one or more agents selected from anti-acne agents, keratolytics, comedolytics, sebostatic/sebosuppressive agents, anti-inflammatories and additional antimicrobial (especially antibacterial) agents. It may instead or in addition contain one or more agents selected from sunscreens, moisturisers and mixtures thereof.

Generally speaking a formulation according to the invention may contain one or more agents which enhance the activity of another active agent present in the formulation, or reduce a side effect of such an active, or improve patient compliance on administration of the formulation. It may contain one or more agents which control the site and/or rate of release of an active agent following administration.

An additional antimicrobial agent may for example be selected from the group consisting of biocides, disinfectants, antiseptics, antibiotics, bacteriophages, enzymes, anti-adhesins, immunoglobulins, other antimicrobially active antioxidants and mixtures thereof. It is preferably active as a bactericide, in particular against propionibacteria, staphylococci and/or bacteria implicated in body odour, more particularly against propionibacteria and/or bacteria implicated in body odour, yet more particularly against propionibacteria.

It may however be preferred for the pyridine thiol and the bis-quinolinium salt to be the only active agents in the formulation, or at least to be the only antimicrobially active agents (or at least the only antibacterially active agents) and/or the only anti-acne active agents.

In the case where the formulation is intended for use in the treatment of body odour, it may contain an anti-perspirant such as an aluminium or aluminium-zirconium salt, and/or a deodorising agent. It may be in the form of an aerosol, or of a roll-on or “stick” deodorant of known type, or of a dusting powder such as a talcum powder, or of a gel or cream or ointment. It may be coated on or incorporated into a shoe insert such as a sock or insole. In each case it may contain appropriate conventional liquid or solid carriers and excipients. It may contain one or more perfumes.

In the case where the formulation is intended for application to a non-living area or surface, for instance as a disinfectant, it may take the form of a solution or suspension of the pyridine thiol and the bis-quinolinium salt in an appropriate fluid vehicle such as an alcohol or a water/alcohol mix. Again conventional excipients and other additives may be included, as may one or more additional antimicrobial (in particular antibacterial) agents.

In particular when the formulation is for use in controlling the transmission of a bacterial infection, it may be in the form of a skin wash (for example a hand wash), or of a surface disinfectant such as a spray, or of a cleansing fluid for example for use in disinfecting surgical instruments. It may be carried in or on a cloth, wipe, brush or other cleaning utensil, or a substrate such as a preparation surface or implement or a packaging material; in such cases an item may be impregnated with, or coated with, the formulation.

Thus a formulation according to the invention may be suitable for, more preferably adapted for, use in an area or on a surface other than living tissue, for instance to treat floors or walls (whether internal or external), work surfaces or instruments, or to cleanse hair or nails so as to reduce microbe levels. It may be suitable for application to non-living tissue (for instance for use as a preservative) or clothing (for instance for bio-agent decontamination). In these cases the excipients, vehicles and/or other additives included with the pyridine thiol and the bis-quinolinium salt may be different to those included in a topical skin care or oral health care formulation, but again may be conventional as known for use in such contexts. In particular the formulation, or either or both of the two active agents, may be provided in the form of a concentrate which can be diluted to a suitable concentration (for example as described above) prior to use.

In some cases it may be suitable for a formulation according to the present invention not to contain a cetyl pyridinium salt, for instance as disclosed in U.S. Pat. No. 3,147,182.

In some cases it may be suitable for a formulation according to the present invention not to contain an imidazole (in particular miconazole nitrate), for instance as disclosed in U.S. Pat. No. 6,001,864.

In some cases it may be suitable for a formulation according to the present invention not to contain an alkanol potentiator, in particular a phenyl alkanol, for instance as disclosed in U.S. Pat. No. 4,474,748.

In some cases it may be suitable for a formulation according to the present invention not to contain both an alcohol and an organic acid as delivery vehicles, for instance as disclosed in U.S. Pat. No. 4,294,852.

In some cases it may be suitable for a formulation according to the present invention not to contain an antibiotic, for instance as described in US-2006/0019987.

In some cases it may be suitable for a formulation according to the present invention not to contain a thio-amino acid or related compound, for instance as disclosed in U.S. Pat. No. 4,176,197.

In some cases it may be suitable for a formulation according to the present invention not to contain a quaternary ammonium compound other than the bis-quinolinium salt, in particular a benzalkonium salt such as benzalkonium chloride.

In cases it may be suitable for a formulation according to the invention not to include an alcohol, alkanol or phenol (in particular cyclohexyl phenol) potentiator, for example of the kind referred to in U.S. Pat. No. 4,006,218.

A formulation according to the invention may be incorporated into, and hence applied in the form of, another product such as a cosmetic; a skin or hair care preparation (for example a skin cleanser, toner or moisturiser, or a shampoo, conditioner, styling mousse or gel or hair spray); a deodorant or anti-perspirant; a cleansing preparation (for example a hand wash for use by surgeons prior to treating patients); a pharmaceutical (which includes veterinary, but is preferably for human use) preparation; a cosmeceutical preparation; a toiletry product (for instance a bath or shower additive or a soap); a laundry or other fabric treatment product or an agricultural or horticultural product.

The invention therefore provides, according to a second aspect, a product which incorporates an antimicrobial formulation according to the first aspect.

A formulation according to the invention may be marketed with an indication that it has antimicrobial activity, or enhanced antimicrobial activity, for example against one or more of the pathogens referred to herein. The marketing of such a formulation may for example include an activity selected from (a) enclosing the formulation in a container or package that comprises the relevant indication; (b) packaging the formulation with a package insert that comprises the indication; (c) providing the indication in a publication that describes the formulation; and (d) providing the indication in a commercial which is aired for instance on the radio, television or internet. The antimicrobial activity of the formulation may be attributed, in such an indication, at least partly to the presence of the combination of the pyridine thiol and the bis-quinolinium salt.

The invention may involve assessing the antimicrobial activity of the formulation during or after its preparation, for instance against one or more of the pathogens referred to herein. It may involve assessing the antimicrobial activity of the formulation both before and after incorporation of the pyridine thiol and/or the bis-quinolinium salt, for example so as to confirm that either or preferably both contribute to the antimicrobial activity of the formulation.

The formulation of the invention may be prepared in situ, at or immediately before its point of use, for instance its application to the skin or another surface. Thus according to a third aspect, the present invention provides a kit for preparing an antimicrobial formulation, for example a formulation according to the first aspect, the kit comprising a source of a pyridine thiol and a source of a bis-quinolinium salt, together with instructions for combining the two compounds so as to make the formulation at or before the point of intended use, and/or for the co-administration of the two compounds to a surface such as the skin. The two compounds may each be present in a suitable respective vehicle.

According to one embodiment, the formulation or kit of the invention may contain both a pyridine thiol and a bis-quinolinium salt, each encapsulated (for instance microencapsulated) in a separate delivery vehicle; this might for instance allow their release, and hence their contact with one another, only at the intended site of administration.

A fourth aspect of the invention provides a method for preparing an antimicrobial formulation, which method involves mixing together a pyridine thiol and a bis-quinolinium salt, preferably together with a pharmaceutically acceptable vehicle.

According to a fifth aspect of the invention there is provided a formulation (preferably a formulation according to the first aspect of the invention) containing a pyridine thiol and a bis-quinolinium salt, for use in the treatment of a condition affecting the human or animal body, which condition is caused by, transmitted by and/or exacerbated by (in particular caused or transmitted by) microbial activity. Again the condition may be caused by, transmitted by and/or exacerbated by (in particular caused or transmitted by) bacterial activity. It may in particular be a skin or skin structure condition such as acne or a skin or skin-borne infection. It may be a staphylococcal infection. It may be body odour.

In the context of the present invention, treatment of a condition encompasses both therapeutic and prophylactic treatment, of either an infectious or a non-infectious condition, in either a human or animal but in particular a human, suitably on the skin. It may involve complete or partial eradication of the condition, removal or amelioration of associated symptoms, arresting subsequent development of the condition, and/or prevention of, or reduction of risk of, subsequent occurrence of the condition. It will typically involve use of the pyridine thiol and the bis-quinolinium salt as a biocidal combination, in particular as a bactericidal combination, more particularly against propionibacteria. It may involve the prophylactic treatment of any area of the body, in particular the skin or nares or ears or another epithelial or mucosal surface, against microbial infections, including against staphylococcal infections such as those associated with MRSA. In cases it may involve use of the pyridine thiol and the bis-quinolinium salt as an antifungal combination.

The treatment of a condition also embraces the prevention, or reduction of risk of, dissemination or transmission of the condition, for example from person to person. In this context, the pyridine thiol and the bis-quinolinium salt may be used in combination as a disinfectant against the relevant micro-organism, for example for antisepsis of the skin and/or other appropriate parts of the body, or for the general disinfection of surfaces in an area believed to be contaminated with, or at risk of contamination with, the organism. Thus the invented combination may be used to treat an outbreak of a particular pathogen, for example a nosocomial pathogen such as S. aureus (including resistant strains such as MRSA, VISA or GISA), E. faecalis or C. difficile.

In the context of the present invention, treatment of a condition may in particular involve use of the formulation against propionibacteria, and/or against Gram-positive cocci such as staphylococci or streptococci, and/or against bacteria associated with body odour.

It may involve use of the formulation against microbial biofilm formation. Thus, the formulation may be used to treat a condition which is caused, transmitted and/or exacerbated by (in particular caused or transmitted by) microbial biofilm formation, in particular biofilm formation which is caused or exacerbated by, or which otherwise involves (in particular which is caused by), bacterial activity.

In an embodiment of the invention, the invented formulation is for use against one or more micro-organisms (in particular bacteria) associated with skin or skin-borne infections. It may be for use against Gram-positive bacteria, for example propionibacteria, in particular against strains of Propionibacterium acnes. In an embodiment, it is for use against one or more bacteria associated with acne, such as P. acnes and in some instances P. granulosum.

According to an embodiment of the fifth aspect of the invention, the formulation is for use in the treatment of a skin or skin structure condition. Such a condition may be a primary or secondary infection. It may for example be a superficial or uncomplicated skin infection amenable to local therapy. It may be acne or an infection associated with acne. It may be a primary or secondary infection due to S. aureus (including MRSA) or a group a beta haemolytic streptococcus (S. pyogenes)). The formulation may in particular be for use in the treatment of acne (ie, as an anti-acne agent).

Skin and skin structure conditions which might be treated according to the invention include acne, infected atopic eczema, superficial infected traumatic lesions, wounds, burns, ulcers, folliculitis, mycoses and other primary and secondary skin and skin structure infections. In particular the invented formulation may be for use in treating acne or acne lesions (for instance, to reduce acne-related scarring).

Acne is a multifactorial disease of the pilosebaceous follicles of the face and upper trunk, characterised by a variety of inflamed and non-inflamed lesions such as papules, pustules, nodules and open and closed comedones. Its treatment can therefore encompass the treatment (which embraces prevention or reduction) of any of these symptoms, and references to use as an anti-acne agent may be construed accordingly.

In particular, the treatment of acne encompasses the treatment (including prevention) of lesions and/or scarring associated with acne. It also encompasses the treatment of a propionibacterial infection and/or the inhibition of propionibacterial activity which could cause or be otherwise associated with acne or its symptoms.

In general, the present invention will be used for the treatment of symptoms which are directly due to acne rather than for instance infections which may arise as a consequence of treating acne with other actives such as antibiotics, and/or secondary infections caused by opportunistic pathogens, which can arise in skin already affected by acne.

Thus, in general terms the invention can provide a formulation containing a pyridine thiol and a bis-quinolinium salt, for use in the treatment of acne.

Atopic dermatitis or eczema (atopic eczema and dermatitis syndrome AEDS), which may also be treated using the present invention, can frequently become infected by Gram-positive bacteria, most commonly by Staphylococcus aureus (David T J, 1989 Journal of the Royal Society of Medicine 82: 420-422) but also by members of the genus Streptococcus (Brook I, 2002 Journal of Medical Microbiology 51: 808-812) and possibly by members of the genus Enterococcus. The invented formulation may be for use in the treatment of infected atopic dermatitis since such combinations of compounds have been shown to be active against S. aureus (including Methicillin Resistant S. aureus (MRSA) and Epidemic Methicillin Resistant S. aureus (EMRSA)), members of the genus Streptococcus and members of the genus Enterococcus (including E. faecalis), and is therefore also expected to be active against Vancomycin-Intermediate Resistant S. aureus (VISA) and Glycopeptide-Intermediate Resistant S. aureus (GISA). The formulation may be for use against one or more such bacteria.

Human skin is susceptible to infection by a wide range of bacteria. These conditions include but are not restricted to folliculitis, boils and carbuncles, impetigo most usually caused by Staphylococcus aureus and other infections including erysipelas caused by members of the genus Streptococcus, erythrasma and cellulitis caused by both staphylococci and streptococci. Other bacteria may also be involved in these infections including members of the genera Bacillus, Clostridium and Enterococcus. According to the invention, the combination of pyridine thiol and bis-quinolinium salt may be for use in the treatment of infected dermatoses, skin infections, superficial infected wounds and soft tissue infections since such combinations of compounds have been shown to be active against S. aureus, including MRSA and EMRSA, as well as against members of the genus Streptococcus, members of the genus Enterococcus (including E. faecalis) and members of the genera Bacillus and Clostridium. The combinations may also be used against VISA and GISA staphylococcal strains, and/or against coagulase-negative staphylococci.

Infected dermatoses, skin infections, superficial infected wounds and soft tissue infections may also include polymicrobial infections of the skin that are caused by both Gram-positive and Gram-negative bacteria. Again such infections may be treated using a formulation according to the present invention.

In an embodiment of the fifth aspect of the invention, the invented formulation may be for use as a treatment against staphylococci, which might otherwise cause for example MRSA-associated infections. It may be for use as a treatment against staphylococci on the skin, or in the nares, eyes or ears. The formulation may in particular be for use in a prophylactic treatment against staphylococci (in particular S. aureus) in the nasal carriage.

Approximately 25 to 30% of healthy individuals carry Staphylococcus aureus in the nares. The organism is also carried at other body sites and at higher prevalence in predisposed individuals such as those with atopic dermatitis. Antibiotic-resistant strains of Staphylococcus aureus (eg, MRSA) are also widely distributed both in the hospital environment and in the community. These factors contribute to the risk of nosocomial S. aureus infections especially in patients undergoing surgery (Grundmann H, Aires-de-Sousa M, Boyce J, Tiemersma E, 2006 Lancet 368: 874-85; Herwaldt L A, 2003 Surgery 134(5 Suppl):S2-9). Combinations of pyridine thiols and bis-quinolinium salts have been shown capable of activity against S. aureus and may be used prophylactically to eradicate and/or prevent colonisation of the nares and skin by this organism. This can be used for example in patients and hospital staff to prevent infections caused by S. aureus. The combinations are particularly well suited for this purpose as they can be active against antibiotic-resistant strains of S. aureus such as MRSA and EMRSA.

According to a further embodiment of the invention, the invented formulation is for use in the treatment (which includes prevention) of body odour, for example in the axilla or feet. It may thus be for use against the bacteria implicated in this condition, in particular aerobic diphtheroids of the genus Corynebacterium.

Human body odour is formed by the action of commensal skin bacteria on the odourless secretions of sweat glands. For example the action of members of the genus Corynebacterium have been shown to release the odiferous compounds 3-hydroxy-3-methylhexonic acid and 3-hydroxy-2-methylhexonic acid from odourless precursors (Natsch A, Gfeller H, Gygax P et al, 2003 Journal of Biological Chemistry 278 (8): 5718-5727). The formulation may thus be for use in the prevention of body odour as such combinations of compounds have been shown to be active against species of the genus Corynebacterium, including corynebacteria of human origin such as C. mucifaciens. Such combinations have also been shown to be active against other members of the bacterial human skin microflora such as cutaneous propionibacteria, which may also contribute to human body odour. They are also expected to be active against coagulase-negative staphylococci which again can contribute to body odour. According to the invention, the combination of a pyridine thiol and a bis-quinolinium salt may therefore be used against one or more such bacteria.

In an embodiment, the invented formulation may be for use in the treatment of an ocular infection. It may for example be used in the treatment of conjunctivitis due to Corynebacterium spp or in particular S. aureus, or in the treatment (in particular the prevention) of endophthalmitis due to Propionibacterium spp.

In an embodiment, the formulation may be for use in the treatment of an infection within the ear.

Infections of the eye and ear are commonly caused by Gram-positive and Gram-negative bacteria (Kowalski R P, Dhaliwal D K, 2005 Expert Rev Anti Infect Ther. 3(1):131-9; de Miguel Martínez I, Ramos Macías A, Masgoret Palau E, 2007 Acta Otorrinolaringol Esp. 58(9):408-12). They are often caused by staphylococci, streptococci and occasionally by cutaneous propionibacteria. According to the fifth aspect of the invention, the invented formulation may be for use in the treatment of infections of the eye or ear since such combinations of compounds have been shown to be active against the following bacteria that are commonly identified as causative agents: Staphylococcus aureus (including MRSA and EMRSA), streptococci, and propionibacteria such as Propionibacterium acnes and Propionibacterium granulosum.

In an embodiment, the invented formulation may be for use in the treatment of an infection of a wound (in particular a deep-seated wound), burn or ulcer. Deep-seated wounds, burns and ulcers are often infected by either Gram-positive bacteria, Gram-negative bacteria or mixed populations containing both types of bacteria (Hedrick T L, Smith P W, Gazoni L M et al, 2007, Current Problems in Surgery 44(10):635-75; Meara S O, Cullum, N, Majid M et al, 2000 Health Technology Assessment 4(21); Church D, Elsayed S, Reid O, 2006 Clinical Microbiology Reviews 19(2): 403-434; Anderson C A, Roukis T S, 2007 Surgical Clinics of North America 87:1149-1177). The invented combinations of compounds have been found capable of activity against a range of Gram-positive bacteria involved in such infections, and against certain anaerobic Gram-negative bacteria which can also be involved in these infections.

The invented formulation may be for use in the treatment of infected deep-seated wounds, burns and ulcers since such combinations of compounds have been shown to be active against the following bacteria that are commonly identified as the infective agents: Staphylococcus aureus (including MRSA and EMRSA), members of the genus Streptococcus, members of the genus Enterococcus (including E. faecalis), members of the genus Bacillus (eg, B. cereus), members of the genus Clostridium, propionibacteria such as Propionibacterium acnes and Propionibacterium granulosum, and members of the genus Corynebacterium (eg, C. mucifaciens).

The invented formulation may be for use in the treatment of a urinary tract infection (UTI). Such infections are caused by either Gram-positive bacteria, Gram-negative bacteria or mixed populations containing both types of bacteria. Among the Gram-positive organisms frequently implicated in UTIs are Staphylococcus aureus, coagulase-negative staphylococci and enterococci. The invented formulation may be for use in the treatment of UTIs since such combinations of compounds have been shown to be active against the following bacteria that are commonly identified as causative agents of UTIs: S. aureus (including MRSA and EMRSA), and members of the genus Enterococcus (including E. faecalis).

In an embodiment, the invented formulation may be for use in the treatment of an infection associated with an indwelling surgical device or implant, for example a catheter. Bacterial infections can frequently arise through the use of such devices (Wenzel R P, 2007 CID 45 (Suppl 1): S85-S88). This is particularly the case when Staphylococcus aureus, coagulase-negative staphylococci, streptococci, cutaneous propionibacteria (eg, in the case of artificial hip joints) and other bacteria adhere to the device and form a focus of infection and/or biofilm. Bacteria can detach from the initial infectious site and may be linked to (ie, may in cases cause, increase susceptibility to and/or exacerbate) other, systemic conditions such as bacteraemia and its sequlae, including for example acute and subacute endocarditis. Thus the invented formulation may be for use in the treatment of such infections and/or associated conditions.

The invented formulation may thus be used in the treatment of infections associated with indwelling surgical devices, since such combinations of compounds have been shown to be active against S. aureus (including MRSA and EMRSA), members of the genus Enterococcus (including E. faecalis), members of the genus Streptococcus, propionibacteria such as Propionibacterium acnes and Propionibacterium granulosum and members of the genus Corynebacterium (including C. mucifaciens).

Infections associated with catheters and other indwelling surgical devices may also be polymicrobial infections caused by both Gram-positive and Gram-negative bacteria. The invented formulation may also be of use in treating these types of infection.

In an embodiment, the invented formulation may be for use in the treatment, in particular the prophylaxis, of an opportunistic infection. Immuno-compromised individuals who are otherwise susceptible to infection, for example due to HIV infection or other underlying diseases, malnutrition or the administration of immunosuppressive drugs, can be predisposed to opportunistic bacterial infections. Such infections can be caused by a wide range of Gram-positive and Gram-negative bacteria. Formulations according to the invention have been found capable of activity against a wide range of bacteria involved in such infections. Thus the invented formulation may be for use in the treatment of opportunistic infections since such combinations of compounds have been shown to be active against the bacteria which are commonly identified as the infective agents, for example: Staphylococcus aureus (including MRSA and EMRSA), members of the genus Streptococcus (including S. mutans), members of the genus Enterococcus (including E. faecalis), members of the genus Bacillus (eg, B. cereus), members of the genus Clostridium (eg, C. difficile), cutaneous propionibacteria such as Propionibacterium acnes and Propionibacterium granulosum, and members of the genus Corynebacterium (including C. mucifaciens). For this purpose the invented formulation may for instance be applied to a dressing, surgical instrument, implant, catheter or the like to reduce the risk of bacterial infection during or after use of the item.

In an embodiment, the invented formulation may be for use in preventing the transmission of a food-borne bacterial pathogen, for example an infection caused by S. aureus, B. cereus, E. faecalis or Listeria monocytogenes. Moreover, as discussed below, the invented formulation may be used to control bacterial growth in order to inhibit, prevent or reduce food spoilage. Such combinations of compounds have been shown to be active, or by inference from experimental data are believed to be active, against the following bacteria that are commonly identified as food spoilage agents and food borne pathogens: Staphylococcus aureus (including MRSA, EMRSA, VISA and GISA), members of the genus Streptococcus, members of the genus Enterococcus (including E. faecalis and Enterococcus faecium), members of the genus Bacillus (eg, B. cereus), members of the genus Clostridium (eg, C. sporogenes), members of the genus Lactobacillus and Listeria monocytogenes (sole species).

In an embodiment, the invented formulation may be for use in the disinfection of skin or other tissue surfaces. Moreover, as described below, it may be used for the disinfection of non-living areas and surfaces. It may in particular be used to counter bacteria of the type referred to above. For example, it may be used to disinfect against the following bacteria: Staphylococcus aureus, members of the genus Enterococcus (including E. faecalis and E. faecium), members of the genus Bacillus (eg, B. cereus), members of the genus Clostridium (eg, C. sporogenes), Listeria monocytogenes, Escherichia coli and Pseudomonas spp.

In an embodiment, the invented formulation may be for use in the treatment of a bacterial condition affecting an epithelial or mucosal surface such as in the nares, scalp, vagina, eyes, ears or oral cavity.

In certain cases, the invented formulation may be for use in the treatment of a polymicrobial or mixed infection. In cases, it may be for use in the treatment of a fungal, in particular micro-fungal, infection, for example an infection or other condition which is caused, transmitted and/or exacerbated by Malassezia furfur or Candida albicans. The formulation may be for use in the treatment of a mixed infection which involves a fungus, in particular a micro-fungus.

According to the fifth aspect of the invention, the formulation of pyridine thiol and bis-quinolinium salt may be prepared in situ, at or immediately before the point of administration. This aspect of the invention thus pertains to any use of a pyridine thiol and a bis-quinolinium salt in the treatment of a microbial condition, in particular acne, the two compounds being administered either simultaneously or sequentially.

According to a sixth aspect, the invention provides the use of a pyridine thiol and a bis-quinolinium salt in the manufacture of a medicament (typically a formulation) for the treatment of a condition which is caused by, transmitted by and/or exacerbated by (in particular caused or transmitted by) microbial activity. Again the condition may be caused by, transmitted by and/or exacerbated by bacterial activity. It may be selected from those listed above in connection with the fifth aspect of the invention. It may be a skin or skin structure condition, in particular acne. It may be a staphylococcal infection. It may be body odour. The pyridine thiol and the bis-quinolinium salt will typically be used as antimicrobial agents, or at least as a combined antimicrobial agent, in the manufacture of the medicament. More typically, they will be used as antibacterial agents, or as a combined antibacterial agent.

The invention further provides, according to a seventh aspect, the use together of a pyridine thiol and a bis-quinolinium salt, as an antimicrobial agent, and/or as an anti-acne agent, or in the manufacture of an antimicrobial or anti-acne formulation.

An eighth aspect provides a method for controlling the growth of a micro-organism, in particular a bacterium, the method comprising applying, to an area or surface which is infected or suspected to be infected or capable of becoming infected with the organism, a combination of a pyridine thiol and a bis-quinolinium salt. Again the two compounds may be applied simultaneously or sequentially. They may be applied in a formulation according to the first aspect of the invention. They may in particular be applied to an area or surface which is infected with the organism.

In this context, “controlling the growth” of a micro-organism embraces inhibiting or preventing its growth, whether completely or partially, as well as killing either completely or partially a culture of the organism. It also embraces reducing the risk of subsequent growth of the organism in or on the area or surface being treated. It may embrace reducing the risk of transmission of the organism from the area or surface being treated to another area or surface and/or living body. The method of the invention may thus be used to treat an existing occurrence of the organism or to prevent a potential subsequent occurrence. Controlling the growth of a micro-organism, in particular a bacterium, may also embrace the disruption and/or suppression of biofilm formation by the organism, as described above.

Again the area or surface to which the pyridine thiol and the bis-quinolinium salt are applied will typically be a surface such as human or animal tissue, in particular the skin, typically of a living human or animal. In this case the invented combination may be applied for therapeutic purposes or for non-therapeutic (eg, purely cosmetic) purposes. Thus the method of the eighth aspect of the invention encompasses a method of treatment of a human or animal patient suffering from or at risk of suffering from a condition which is caused by, transmitted by and/or exacerbated by (in particular caused or transmitted by) microbial activity, the method involving administering to the patient a therapeutically (which term includes prophylactically) effective amount of an antimicrobial formulation containing a pyridine thiol and a bis-quinolinium salt. Again the microbial condition may be any of those referred to above in connection with the fifth aspect of the invention, in particular one which is caused, transmitted and/or exacerbated by bacterial activity. The condition may in particular be a skin or skin structure condition such as acne. The antimicrobial formulation is suitably administered in an antimicrobially effective amount.

Alternatively the pyridine thiol and the bis-quinolinium salt may be applied to a non-living area or surface such as in a hospital, dental surgery or food preparation area. For example the method of the eighth aspect of the invention may be used to treat work surfaces, surgical or other instruments, surgical implants or prostheses, protective clothing such as surgical gloves, contact lenses, foods, crops, industrial plant, floors or walls (both internal and external), clothing, bedding, furniture and many other surfaces.

The method of the eighth aspect of the invention preferably involves applying a formulation according to the first aspect.

A ninth aspect of the invention provides the use of a pyridine thiol in an antimicobial or anti-acne formulation, in combination with a bis-quinolinium salt, for the purpose of increasing the antimicrobial and/or anti-acne activity of the formulation and/or of reducing the amount of the bis-quinolinium salt in the formulation without undue loss of antimicrobial or anti-acne activity.

An increase in antimicrobial or anti-acne activity may be as compared to that of the bis-quinolinium salt alone, at the same concentration as used when combined with the pyridine thiol. Ideally the increase is as compared to the sum of the activities of the pyridine thiol and the bis-quinolinium salt individually, again at the same respective concentrations as used when the two are combined.

A reduction in the amount of the bis-quinolinium salt in the formulation may be as compared to the amount which would otherwise have been used in the formulation in order to achieve a desired level of activity, in particular in order to have acceptable efficacy in the context of its intended use. The reduction may be manifested by reduced side effects which would otherwise have been observed during use of the formulation, for example local irritation and/or undesirable systemic absorption of the bis-quinolinium salt. According to the invention, the pyridine thiol may therefore be used for the dual purposes of reducing an undesired property of a formulation containing a bis-quinolinium salt, without undue loss of antimicrobial or anti-acne activity.

Preferably the pyridine thiol is used without any reduction in antimicrobial or anti-acne activity compared to the level exhibited by the formulation prior to addition of the pyridine thiol. More preferably it is used to give an increase in antimicrobial or anti-acne activity. It may however be used to reduce the amount of the bis-quinolinium salt present, and/or its associated side effects, whilst maintaining the antimicrobial or anti-acne activity of the resultant formulation at a level, albeit lower than that which it would otherwise have exhibited, which is still acceptable in the context of its intended use.

A tenth aspect of the invention provides the use of a bis-quinolinium salt in an antimicrobial or anti-acne formulation, in combination with a pyridine thiol, for the purpose of increasing the antimicrobial and/or anti-acne activity of the formulation and/or of reducing the amount of the pyridine thiol in the formulation without undue loss of antimicrobial or anti-acne activity.

An increase in antimicrobial or anti-acne activity may be as compared to that of the pyridine thiol alone, at the same concentration as used when combined with the bis-quinolinium salt. Ideally the increase is as compared to the sum of the activities of the bis-quinolinium salt and pyridine thiol individually, again at the same respective concentrations as used when the two are combined.

A reduction in the amount of the pyridine thiol in the formulation may be as compared to the amount which would otherwise have been used in the formulation in order to achieve a desired level of activity, in particular in order to have acceptable efficacy in the context of its intended use. The reduction may be manifested by reduced side effects which would otherwise have been observed during use of the formulation, for example local irritation and/or undesirable systemic absorption of the pyridine thiol. According to the invention, the bis-quinolinium salt may therefore be used for the dual purposes of reducing an undesired property of a formulation containing a pyridine thiol, without undue loss of antimicrobial or anti-acne activity.

Preferably the bis-quinolinium salt is used without any reduction in antimicrobial or anti-acne activity compared to the level exhibited by the formulation prior to addition of the bis-quinolinium salt. More preferably it is used to give an increase in antimicrobial or anti-acne activity. It may however be used to reduce the amount of the pyridine thiol present, and/or its associated side effects, whilst maintaining the antimicrobial or anti-acne activity of the resultant formulation at a level, albeit lower than that which it would otherwise have exhibited, which is still acceptable in the context of its intended use.

According to an eleventh aspect, the invention provides a method of increasing the antimicrobial and/or anti-acne activity of a formulation, by adding to the formulation a pyridine thiol and a bis-quinolinium salt. The formulation may for example be a pharmaceutical formulation, typically one which is suitable and/or adapted and/or intended for topical application. The combination of the pyridine thiol and the bis-quinolinium salt is suitably added in an antimicrobially effective amount. In accordance with the eleventh aspect of the invention, a pyridine thiol may be added to a formulation which already contains a bis-quinolinium salt, in order to increase the antimicrobial and/or anti-acne activity of the formulation. Similarly, a bis-quinolinium salt may be added to a formulation which already contains a pyridine thiol.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith.

Moreover unless stated otherwise, any feature disclosed herein may be replaced by an alternative feature serving the same or a similar purpose.

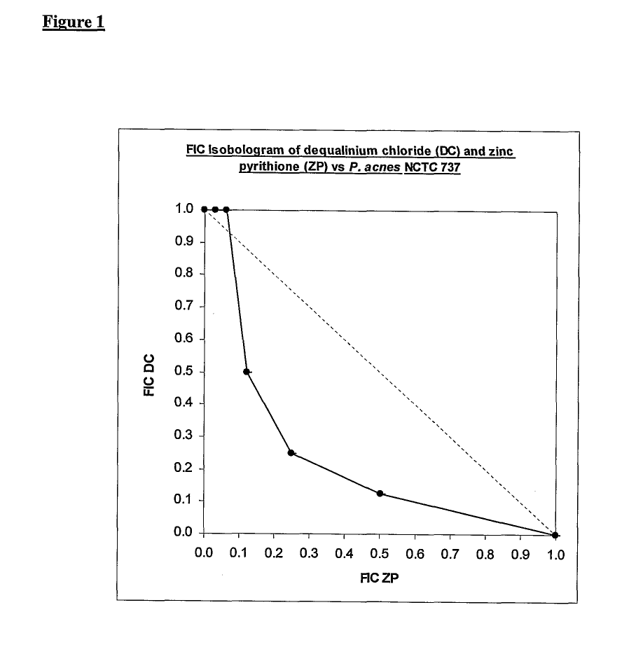

The present invention will now be further described with reference to the following non-limiting examples and the accompanying FIG. 1, which is an isobologram showing FIC (fractional inhibitory concentration) values for mixtures of zinc pyrithione and dequalinium chloride against a propionibacterial strain, as referred to in Example 2 below.

### DETAILED DESCRIPTION

Experimental tests were conducted to determine the antibacterial activity of formulations according to the invention. As a comparison, the antibacterial activities of formulations containing a pyridine thiol or a bis-quinolinium salt alone were also measured.

Test Micro-Organisms

The first test micro-organism used was a propionibacterial strain, Propionibacterium acnes NCTC 737. This is the type strain of the genus; it is fully susceptible to antibiotics.

The propionibacteria are clinically significant due to their involvement in acne, which is a very common, complex and multi-factorial skin disease in which P. acnes and other Propionibacterium spp. (for example P. granulosum) play key roles. They are also opportunistic pathogens in compromised hosts. Thus, activity observed against these micro-organisms is expected to be a good predictor of activity against acne.

Other propionibacterial strains were also tested, as described in Example 4 below. These included certain antibiotic resistant propionibacteria, such as the two P. acnes strains designated PRP-010 and PRP-039 which are resistant respectively to macrolides-lincosamides-streptogramins-ketolides (MLSK) and to macrolides-lincosamides-streptogramins (MLS) and tetracycline—in other words, PRP-010 is resistant to erythromycin and clindamycin, and PRP-039 to erythromycin, clindamycin and tetracycline.

In addition, certain strains of P. granulosum, another bacterium involved in acne, were also tested in Example 4.

The propionibacteria were cultured and maintained on Wilkins-Chalgren Anaerobe Medium (agar and broth) at pH 6.0; all cultures were incubated anaerobically at 37° C. for 72 hours.

The following additional test organisms were also used:

| | | Culture conditions | | | |
|---|---|---|---|---|---|
| | Gram | Growth | Temp | | Time |
| Test organism | test | medium | (° C.) | Atmosphere | (h) |
| Clostridium difficile | + | WC pH7 + | 37 | Anaerobic | 48 |
| ATCC 7000057 | | 5% lysed | | | |
| | | horse | | | |
| | | blood | | | |
| Corynebacterium | + | MH | 37 | Aerobic | 24 |
| mucifaciens | | | | | |
| ATCC 700355 | | | | | |

[Abbreviations: American Type Culture Collection (ATCC), Mueller-Hinton (MH), Wilkins-Chalgren (WC).]

Clostridium difficile is the most significant cause of Pseudomembranous colitis, an infection of the colon usually associated with the normal gut flora being eradicated during antibiotic treatment. Corynebacterium mucifaciens is closely related to organisms (aerobic diphtheroids of the genus Corynebacterium) that cause body odour.

The following tests were carried out to assess antimicrobial activity against the test organisms.

(a) Minimum Inhibitory Concentration (MIC) Assay

This is a standard international method for quantitatively assessing the antimicrobial activity of a compound in a liquid medium. The method used a sterile 96-well microtitre plate, capable of holding about 200 μl of liquid per well. The wells contained liquid culture medium and ranges of decreasing concentrations of the relevant test compound in doubling dilutions (eg, 1000, 500, 250, 125 . . . μg/ml, etc. down to 0.49 μg/ml). The culture medium was as described above.

The wells were inoculated with a liquid suspension of freshly grown micro-organism and incubated under the conditions described above. After incubation, the microtitre plate was examined visually (with the aid of a light box) for cloudiness in each well, which would indicate microbial growth. The MIC value was recorded as the lowest concentration of test compound required to inhibit microbial growth, ie, the lowest concentration for which the liquid in the well remained clear.

The assays were conducted in duplicate and included both negative (culture medium with no micro-organisms) and positive (culture medium plus diluting solvent plus micro-organism) controls.

Since inhibition does not necessarily indicate killing of microbial cells, merely that growth as visible to the naked eye has been inhibited, it is desirable to conduct a further test (the MBC assay described below) to establish the concentration of the test compound needed to kill the test organism.

(b) Minimum Bactericidal Concentration (MBC) Assay

This assay, normally carried out after an MIC assay, determines the minimum concentration of a compound that is lethal to the micro-organism being tested.

Following an MIC assay, a 5 μl sample was withdrawn from the first microtitre well that showed positive growth and from all the subsequent wells that showed no growth. These samples were then individually sub-cultured on antibiotic-free agar medium, under the incubation conditions described above. Following incubation they were examined visually for bacterial growth. The MBC was taken to be the lowest test compound concentration for which the incubated sample showed no growth.

The ratio of MIC to MBC should ideally be as close to 1 as possible. This facilitates selection of the lowest possible effective concentration of a test compound with a reduced risk of selecting a sub-lethal concentration which could promote resistance or allow the target bacterial population to recover.

(c) Disc Diffusion Assay (DDA)

This is an internationally recognised standard method for qualitatively assessing the antimicrobial activity of a compound.

A sterile paper disc was impregnated with a sample of the test compound in a suitable solvent and 30 minutes allowed for the solvents to evaporate (where possible). The disc was then placed on an agar plate onto which the test micro-organism had been inoculated. The plate was then incubated under the conditions described above, following which it was examined visually for signs of microbial growth. If the test compound had antimicrobial activity, a circular zone of no growth would be obtained around the disc. The diameter of this zone of “inhibition” was measured using a ProtoCOL™ automated zone sizer (Synbiosis, Cambridge, UK). In general, a greater diameter and/or area of the zone of inhibition indicates a greater antimicrobial activity in the relevant test compound, although other factors such as test compound mobility through the agar gel may also influence the result.

(d) Synergy Disc Diffusion Assay (SDDA)

This is a variation on the DDA method, in which two compounds are tested together for their combined antimicrobial activity.

Two test compounds A and B were placed on a single paper disc and the above described DDA procedure repeated. An increase in diameter of the zone of inhibition, compared to the greater of the zone diameters for the two compounds individually, was taken to indicate potential antimicrobial synergy. In practical terms, an increase of greater than 5 mm could be treated as significant.

(e) Fractional Inhibitory Concentration (FIG) Assay

This assay was used to determine the mode of interaction between two antimicrobial test compounds A and B. It was similar to the MIC assay, utilising a 96-well microtitre plate and liquid culture medium. The test compounds were added together to each well at a range of concentrations starting at their respective MIC values and descending in doubling dilutions as with the MIC assay. Typically an 8×8 array of wells could be used to combine 8 different concentrations of compound A (from its MIC downwards, including zero) with 8 different concentrations of compound B (ditto).

The wells were inoculated with freshly grown micro-organism and incubated under the conditions described above.

As for the MIC assay, the results were read by the naked eye. A minimum inhibitory concentration was recorded for each combination of A and B. A fractional FIC index (FICI) was then calculated for each compound in that mixture, and these two indices were added together to give an overall FICI indicative of the mode of interaction.

Thus for each mixture tested, the FIC for compound A (FIC_{A})=MIC for (A+B)/MIC for A alone. Similarly the FIC for compound B (FIC_{B})=MIC for (A+B)/MIC for B alone. The overall FICI=FIC_{A}+FIC_{B}.

An FICI of 0.5 or less was taken to indicate synergy, a value from 0.5 to 4.0 an indifferent effect and values greater than 4.0 antagonism (ie, the two compounds counter one another's activity, leading overall to a diminished antimicrobial effect) (see Odds F C, “Synergy, antagonism, and what the chequerboard puts between them”, J Antimicrob Chemother, 2003; 52: 1). These results can be depicted visually on a plot (isobologram) of FIC_{A} against FIC_{B} for the mixtures tested.

### Example 1

### Activity Against P. acnes (MIC, MBC & Disc Diffusion Assays)

The following experiments all used P. acnes NCTC 737 as the test organism.

MIC, MBC and DDA assays, as described above, were carried out using the test compounds (a) dequalinium chloride (DC), dissolved in ethanol and (b) zinc pyrithione (ZP), dissolved in DMSO. Both compounds were sourced from Sigma-Aldrich, UK.

Mixtures of the two test compounds were then subjected to SDDA assays as described above. Increases in zone diameter (mm) were measured with respect to the DC, which was the compound showing the larger zones of inhibition during the previous individual disc diffusion assays.

For the (S)DDA experiments, 50 μg of the DC was loaded onto each disc, and 200 μg of the ZP. All the (S)DDA experiments were conducted in triplicate.

The MIC and MBC results are shown in Table 1 below and the (S)DDA results in Table 2. All results are collated from a number of experiments.

TABLE 1

| Test compound | MIC (μg/ml) | MBC (μg/ml) | MIC/MBC ratio |
|---|---|---|---|
| DC | 7.8 | 15.6 | 0.5 |
| ZP | 0.98 | 7.8 | 0.125 |

TABLE 2

| Test DDA | | SDDA with DC | SDDA increase | SDDA area |
|---|---|---|---|---|
| compound | (mm) | (mm) | (mm) | increase (%) |
| DC | 16.68 | | | |
| | (±0.47) | | | |
| ZP | 15.85 | 45.39 | 28.7 | 640.1 |
| | (±1.42) | (±5.17) | | |

These data show that both zinc pyrithione and dequalinium chloride alone are active against P. acnes NCTC 737.

Surprisingly, however, when the two compounds are combined the data indicate a synergistic antibacterial interaction between them, with a significant increase in zone diameter and area over those exhibited by either compound alone.

### Example 2

### Activity Against P. acnes spp (FIC Assays)

Mixtures of dequalinium chloride and zinc pyrithione, containing various relative proportions of the two actives, were then subjected to FIC assays against P. acnes NCTC 737, as described above. The results were used to prepare FIC isobolograms. All assays were conducted in triplicate.

The overall FICI obtained for the mixtures was 0.5, representing the mean of three replicates. This indicates a synergistic interaction. A representative isobologram is shown in FIG. 1; the dashed line indicates where overall FICIs (ie, FIC_{DC}+FIC_{ZP}) equal 1, which would indicate a purely indifferent interaction. FIG. 1 clearly demonstrates the synergistic activity of the combination of the pyrithione and the bis-quinolinium salt against P. acnes NCTC 737.

### Example 3

### Activity Against P. acnes (Another Bis-Quinolinium Salt)

The general method of Example 1 was repeated using another bis-quinolinium salt, dequalinium iodide (DI), which was also obtained from Sigma-Aldrich, UK. This compound was tested against P. acnes NCTC 737, both alone and in combination with ZP.

For the (S)DDA experiments, 50 μg of the DI was loaded onto each disc, and 200 μg of the ZP. The DI and ZP were both dissolved in DMSO. Increases in zone diameter (mm) were measured with respect to the ZP, which was the compound showing the larger zones of inhibition during the previous individual disc diffusion assays.

All the (S)DDA experiments were conducted in triplicate.

The MIC and MBC results are shown in Table 3 below and the (S)DDA results in Table 4. All results are collated from a number of experiments.

TABLE 3

| Test compound | MIC (μg/ml) | MBC (μg/ml) | MIC/MBC ratio |
|---|---|---|---|
| DI | 3.9 | 125 | 0.03 |

TABLE 4

| Test | | SDDA with | SDDA increase | SDDA area |
|---|---|---|---|---|
| compound | DDA (mm) | DI (mm) | (mm) | increase (%) |
| DI | 21.92 | | | |
| | (±0.18) | | | |
| ZP | 46.44 | 63.90 | 17.45 | 89.29 |
| | (±4.59) | (±5.19) | | |

These data show that the DI alone is active against P. acnes NCTC 737. Surprisingly, however, when the DI is combined with ZP the data indicate a synergistic antibacterial interaction between them, with a significant increase in zone diameter and area over those exhibited by either compound alone.

### Example 4

### Activity Against Other Propionibacteria spp

Using DC and ZP as the test compounds, the activity of each compound alone and in combination was determined, using (S)DDA tests, against a number of other Propionibacterium spp strains. Some of these strains have known antibiotic resistance.

50 μg of the DC and/or 200 μg of the ZP were loaded onto each disc. All the experiments were conducted in triplicate.

The results are shown in Table 5 below; the resistance phenotype for each of the test strains is indicated.

TABLE 5

| | | | | SDDA | | SDDA |
|---|---|---|---|---|---|---|
| | | DDA | DDA | DC + | SDDA | area |
| | Resistance | DC | ZP | ZP | increase | increase |
| Test organism | phenotype | (mm) | (mm) | (mm) | (mm) | (%) |
| P. granulosum NCTC 11865 | None | 14.08 | 23.95 | 39.36 | 15.42 | 170.20 |
| | | (±1.08) | (±2.31) | (±11.05) | | |
| P. acnes PRP-002 | Tet/MLS | 20.04 | 25.79 | 42.96 | 17.16 | 177.34 |
| | | (±1.93) | (±1.52) | (±3.55) | | |
| P. acnes PRP-003 | Tet | 21.27 | 26.82 | 33.91 | 7.09 | 59.86 |
| | | (±0.92) | (±8.21) | (±4.85) | | |
| P. acnes PRP-004 | Tet | 20.45 | 12.43 | 31.34 | 10.89 | 134.91 |
| | | (±1.81) | (±0.64) | (±1.25) | | |
| P. granulosum PRP-005 | MLSK | 20.55 | 20.04 | 26.00 | 5.45 | 60.02 |
| | | (±0.47) | (±0.82) | (±1.46) | | |
| P. granulosum PRP-006 | MLS | 26.82 | 32.58 | 36.89 | 4.32 | 28.25 |
| | | (±0.31) | (±3.44) | (±0.94) | | |
| P. acnes PRP-007 | Clin | 18.91 | 10.79 | 23.43 | 4.52 | 53.54 |
| | | (±0.78) | (±0.62) | (±0.82) | | |
| P. acnes PRP-008 | Clin | 19.73 | 14.90 | 27.75 | 8.02 | 97.76 |
| | | (±0.82) | (±1.08) | (±0.31) | | |
| P. acnes PRP-010 | MLSK | 17.37 | 20.96 | 36.17 | 15.21 | 197.73 |
| | | (±0.36) | (±2.16) | (±0.94) | | |
| P. acnes PRP-017 | MLS | 17.27 | 19.01 | 27.95 | 8.94 | 116.17 |
| | | (±1.07) | (±2.93) | (±1.58) | | |
| P. granulosum PRP-019 | MLSK | 18.91 | 19.73 | 27.34 | 7.60 | 91.94 |
| | | (±0.36) | (±1.85) | (±2.49) | | |
| P. granulosum PRP-021 | MLS | 27.95 | 29.70 | 36.17 | 6.47 | 48.35 |
| | | (±0.47) | (±3.21) | (±1.25) | | |
| P. acnes PRP-023 | MLSK | 19.32 | 17.37 | 30.73 | 11.41 | 152.95 |
| | | (±1.98) | (±1.55) | (±2.57) | | |
| P. acnes PRP-026 | MLS | 19.01 | 16.24 | 28.26 | 9.25 | 120.96 |
| | | (±0.64) | (±0.99) | (±2.76) | | |
| P. acnes PRP-039 | Tet/MLS | 28.98 | 29.70 | 32.89 | 3.19 | 22.60 |
| | | (±1.34) | (±7.23) | (±1.46) | | |
| P. granulosum PRP-043 | MLS | 23.02 | 43.37 | 44.81 | 1.44 | 6.75 |
| | | (±2.62) | (±3.65) | (±2.62) | | |
| P. granulosum PRP-044 | MLS | 17.37 | 18.50 | 31.86 | 13.36 | 196.60 |
| | | (±0.18) | (±0.31) | (±0.94) | | |
| P. acnes PRP-046 | None | 21.38 | 35.56 | 46.86 | 11.30 | 73.69 |
| | | (±1.46) | (±2.47) | (±1.88) | | |
| P. acnes PRP-053 | Tet/MLS | 21.38 | 29.91 | 30.93 | 1.03 | 6.99 |
| | | (±2.31) | (±5.61) | (±0.78) | | |
| P. granulosum PRP-055 | None | 29.08 | 50.05 | 54.16 | 4.11 | 17.10 |
| | | (±1.70) | (±0.78) | (±1.70) | | |
| P. acnes PRP-059 | MLS | 19.42 | 16.34 | 27.13 | 7.71 | 95.11 |
| | | (±1.60) | (±0.31) | (±0.31) | | |
| P. acnes PRP-068 | Ery | 20.45 | 14.90 | 34.53 | 14.08 | 185.09 |
| | | (±0.78) | (±1.52) | (±1.11) | | |
| P. acnes PRP-101 | Tet/MLS | 16.96 | 14.39 | 33.30 | 16.34 | 285.59 |
| | | (±0.82) | (±0.64) | (±0.82) | | |
| P. acnes PRP-102 | Tet/MLS | 21.68 | 16.85 | 29.80 | 8.12 | 88.90 |
| | | (±0.99) | (±0.47) | (±1.70) | | |

[Abbreviations: American Type Culture Collection (ATCC), National Collection of Type Cultures (NCTC), Propionibacterium Panel Number (PRP), Tetracycline (Tet), Erythromycin (Ery), Clindamycin (Clin), Macrolide-Lincosamide-Streptogramin (MLS), Macroliode-Lincosamide-Streptogramin-Ketolide (MLSK).]

When the DC and ZP are combined, the synergistic interaction initially observed with P. acnes NCTC 737 was similarly observed, to a greater or lesser degree, against all of the propionibacteria tested. This indicates the utility of the combination either to treat or to prevent infections associated with such bacteria, in particular acne. These results are likely to be of particular clinical value for the antibiotic resistant test strains.

### Example 5

### Topical Anti-Acne Formulations

The results from Examples 1 to 4 show that the combination of a pyridine thiol and a bis-quinolinium salt can be an effective antibacterial agent, in particular against the bacteria associated with acne, with a synergistic impact on the antibacterial activity of the combination compared to those of the individual compounds alone. This can be of use in preparing antibacterial formulations, in particular for topical application to the skin, for either prophylactic or therapeutic use in any context where such bacteria are thought to be involved as possible sources of infection.

Even in cases where a combination of a pyridine thiol and a bis-quinolinium salt has an additive, as opposed to synergistic, antibacterial activity compared to that of the individual compounds, this can be of considerable benefit when preparing formulations for topical use. One of the compounds may be used to replace a proportion of the other, thus lowering any side effects and/or other undesirable properties of the combination without undue loss of antibacterial activity.

A topical formulation for use in treating acne may for example be prepared by combining a pyridine thiol, in particular zinc pyrithione, with a bis-quinolinium salt, in particular a dequalinium salt, in a suitable fluid vehicle and optionally together with conventional additives. Such vehicles and additives may be for instance as found in Williams' “Transdermal and Topical Drug Delivery”, Pharmaceutical Press, 2003 and other similar reference books, and/or in Rolland A et al, “Site-specific drug delivery to pilosebaceous structures using polymeric microspheres”, Pharm. Res. 1993, 10: 1738-44; Mordon S et al, “Site-specific methylene blue delivery to pilosebaceous structures using highly porous nylon microspheres: an experimental evaluation”, Lasers Surg. Med. 2003, 33: 119-25; and Alvarez-Roman R et al, “Skin penetration and distribution of polymeric nanoparticles”, J. Controlled Release 2004, 99: 53-62.

The formulation may be prepared and administered using known techniques. It may for example take the form of a cream, lotion or gel.

The concentrations of the two active agents may be in the ranges described above, and will be determined based on the intended use of the formulation, its intended mode of administration and the activities of the particular chosen active agents.

### Example 6

### Activity Against Other Micro-Organisms

The activity of a DC/ZP combination was determined, using (S)DDAs, against two further test micro-organisms, namely C. difficile ATCC 7000057 and Cor. mucifaciens ATCC 700355. A DI/ZP combination was also tested against Cor. mucifaciens.

In these tests, 50 μg (for C. difficile) or 200 μg (for Cor. mucifaciens) of the DC was loaded onto each disc. The DI and the ZP were both loaded at 200 μg per disc. All the (S)DDA experiments were conducted in triplicate.

The results are shown in Tables 6 (DC+ZP) and 7 (DI+ZP) below. All results are collated from a number of experiments.

TABLE 6

| | | | SDDA | | SDDA |
|---|---|---|---|---|---|
| | DDA | DDA | DC + | SDDA | area |
| | DC | ZP | ZP | increase | increase |
| Test organism | (mm) | (mm) | (mm) | (mm) | (%) |
| C. difficile ATCC | 0.0 | 20.31 | 25.77 | 5.46 | 61.04 |
| 7000057 | (±0.0) | (±0.18) | (±1.09) | | |
| Cor. mucifaciens ATCC | 17.2 | 28.14 | 31.17 | 3.03 | 22.66 |
| 700355 | (±0.31) | (±3.80) | (±0.36) | | |

TABLE 7

| | | | | | SDDA |
|---|---|---|---|---|---|
| | DDA | DDA | SDDA | SDDA | area |
| | DI | ZP | DI + ZP | increase | increase |
| Test organism | (mm) | (mm) | (mm) | (mm) | (%) |
| Cor. mucifaciens ATCC | 22.93 | 28.14 | 36.17 | 8.03 | 62.20 |
| 700355 | (±0.36) | (±3.80) | (±0.48) | | |

Tables 6 and 7 show that a bis-quinolinium salt can be combined with a pyrithione to give a synergistic level of activity against the two clinically important Gram-positive (aerobic and anaerobic) test bacteria. This in turn indicates the likely utility of such combinations in treating bacterial infections caused by these micro-organisms.

### Example 7

### Topical Anti-BO Formulations

Example 6 shows that a combination of a bis-quinolinium salt and a pyridinethiol can be active against bacteria associated with body odour. This can be of use in preparing antibacterial formulations, in particular for topical application to the skin, for prophylactic or therapeutic use in any context where such bacteria are thought to be involved as possible sources of infection. More specifically, it can be of use in preparing formulations for use against body odour, in particular in the axilla and/or feet, again suitably for topical use.

Even in cases where a combination of a pyridine thiol with a bis-quinolinium salt has an additive, as opposed to synergistic, antibacterial activity compared to that of the individual compounds, this can be of considerable benefit when preparing formulations for topical use. One of the compounds may be used to replace a proportion of the other, thus lowering any side effects and/or other undesirable properties of the combination without undue loss of antibacterial activity.

A topical formulation for use in the treatment of body odour may be prepared by formulating a pyridine thiol or a pharmaceutically acceptable derivative thereof, for example a pyrithione such as zinc pyrithione, with a bis-quinolinium salt such as a dequalinium salt, in a suitable fluid vehicle and optionally together with conventional additives, as described above.

The formulation may be prepared and administered using known techniques. It may for example take the form of a roll-on, spray or “stick” anti-perspirant or deodorant formulation, or of a dusting powder such as a talcum powder, or of a gel or cream or ointment. It may contain an anti-perspirant and/or deodorant agent, and/or a fragrance. It may be coated on or incorporated into a sock or shoe, or a shoe insole.

1. An antimicrobial formulation containing a pyridine thiol and a bis-quinolinium salt which contains a cation of formula (I) below: 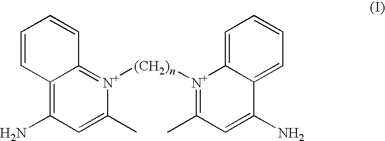
wherein n is an integer from 3 to 18.

2. A formulation according to claim 1, which is suitable for topical application to human skin.

3-46. (canceled)

47. A formulation according to claim 1, wherein the pyrithione is present in the form of a metal pyrithione salt.

48. A formulation according to claim 1, wherein the bis-quinolinium salt is a dequalinium salt.

49. A formulation according to claim 1, wherein the bis-quinolinium salt is a halide.

50. A formulation according to claim 1, which is in the form of a cream, paste, gel, ointment, lotion, foam or other viscous or semi-viscous fluid.

51. A formulation according to claim 1, which is in the form of a spray or dropping fluid.

52. A formulation according to claim 1, which is in the form of an aerosol, roll-on or stick deodorant or anti-perspirant formulation, or a dusting powder, or is coated on or incorporated into a shoe or shoe insert.

53. A product containing an antimicrobial formulation according to claim 1.

54. A kit for preparing an antimicrobial formulation, the kit comprising a source of a pyridine thiol and a source of a bis-quinolinium salt, together with instructions for combining the two compounds so as to make the formulation at or before the point of its intended application, and/or for the co-administration of the two compounds to a surface.

55. A method of treatment of a human or animal patient suffering from or at risk of suffering from a condition which is caused by, transmitted by and/or exacerbated by microbial activity, the method involving administering to the patient a therapeutically (which term includes prophylactically) effective amount of an antimicrobial formulation containing a pyridine thiol and a bis-quinolinium salt.

56. A method according to claim 55, wherein the condition is caused by, transmitted by and/or exacerbated by bacterial activity.

57. A method according to claim 55, wherein the condition is acne and/or acne lesions.

58. A method according to claim 55, wherein the condition is body odour.

59. A method for controlling the growth of a micro-organism, the method comprising applying, to an area or surface which is infected or suspected to be infected or capable of becoming infected with the organism, a combination of a pyridine thiol and a bis-quinolinium salt.
